(19) United States
(12) Patent Application Publication
(10) Pub. No.: US 2002/0058633 A1
(43) Pub. Date: May 16, 2002

(54) Compositions based on glycoglyceride and methods of use therefor, in particular cosmetic uses

(75) Inventor: Odile Sonneville-Aubrun, Antony (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'Oreal, Paris (FR)

(21) Appl. No.: 09/956,429

(22) Filed: Sep. 20, 2001

(30) Foreign Application Priority Data
Sep. 21, 2000 (FR) 0012043

(51) Int. Cl. A61K 38/00; A61K 31/7012; A61K 31/66; A61K 31/195

(52) U.S. Cl. 514/25; 514/102; 514/2; 514/566; 514/23

(57) ABSTRACT

The invention provides compositions for topical use, comprising at least one glycoglyceride and at least one sequestering agent. The glycoglyceride has very good stability in the composition of the invention. The glycoglyceride is preferably a galactolipid such as digalactosyl diglyceride or monogalactosyl diglyceride. The sequestering agent may be chosen in particular from phosphonic derivatives and salts thereof. This composition may in particular constitute a cosmetic and/or dermatological composition.

### CROSS REFERENCES TO RELATED APPLICATIONS

This application claims priority to French Patent Application No. 0012043, filed on Sep. 21, 2000, and which is incorporated herein by reference in its entirety.

### BACKGROUND OF THE INVENTION

1. Field of the Invention

The present invention relates to stable compositions for topical use, which comprise at least one glycoglyceride and at least one sequestering agent, and to the uses, in particular in cosmetics and dermatology, of such compositions.

2. Discussion of the Background

It is a known practice to use natural glycoglycerides, and among these galactolipids, in cosmetic and dermatological compositions. Thus, published European patent applications EP-A-9842 and EP-A-249 561 describe the use of galactolipids to prepare liposomes, and published PCT application WO-A-95/20945 describes the use of digalactosyl diglycerides in combination with apolar lipids such as triacylglycerol, to prepare lipophilic supports which may be used in the pharmaceutical, cosmetic and agrifood fields.

However, compositions which contain natural glycoglycerides have the drawback of changing over time, this change being reflected by very pronounced yellowing of the composition and a development of a very strong rancid odor. These changes in color and odor are unacceptable to users.

Attempts have been made to overcome this drawback by adding, to compositions containing a natural glycoglyceride, antioxidants conventionally used for stabilizing lipids, such as dibutylhydroxytoluene (BHT) or tocopheryl acetate. However, the addition of such antioxidants does not solve the problems of yellowing and rancidity of these compositions.

Thus, there remains a need for glycoglyceride compositions which are stable over time and which in particular do not turn yellow or rancid over time.

### SUMMARY OF THE INVENTION

Accordingly, it is one object of the present invention to provide novel compositions which contain a glycoglyceride.

It is another object of the present invention to provide novel compositions which contain a glycoglyceride and which exhibit a reduced tendency to develop a yellow color over time.

It is another object of the present invention to provide novel compositions which contain a glycoglyceride and which exhibit a reduced tendency to develop a rancid odor over time.

It is another object of the present invention to provide novel methods for treating and/or protecting and/or making up the skin, hair, nails, and mucus membranes with such compositions.

It is another object of the present invention to provide novel methods for stabilizing compositions which contain a glycoglyceride.

These and other objects, which will become apparent during the following detailed description, have been achieved by the inventor's surprising discovery that sequestering agents can stabilize compositions which contain glycoglycerides and, in particular, prevent them from turning rancid and yellow.

Although it is common practice to use sequestering agents in addition to an antioxidant mixture to prevent the oxidation of lipids and to improve their efficacy, it was not obvious that the use of a sequestering agent or a mixture of sequestering agents alone, in the absence of an antioxidant, would block the yellowing of glycoglycerides, the oxidation of the lipid part not being the primary cause of the yellowing since no pentane, which is an indicator of oxidation of unsaturated lipids, is detected.

Accordingly, in a first embodiment, the present invention provides a composition for topical use, comprising in a physiologically acceptable medium, at least one glycoglyceride and at least one sequestering agent.

In another embodiment, the present invention provides methods for treating, protecting, caring for, removing make-up from and/or cleansing the skin, the lips and/or the hair, and/or for making up the skin and/or the lips, comprising applying to the skin, the lips and/or the hair of a subject in need thereof an effective amount of a composition, comprising in a physiologically acceptable medium, at least one glycoglyceride and at least one sequestering agent.

In another embodiment, the present invention provides a method of stabilizing a composition for topical use which contains at least one glycoglyceride, by adding at least one sequestering agent to the composition which contains at least one glycoglyceride.

### DETAILED DESCRIPTION OF THE PREFERRED EMBODIMENTS

As noted above, in a first embodiment, present invention provides a composition for topical use, comprising in a physiologically acceptable medium, at least one glycoglyceride and at least one sequestering agent.

In the context of the present invention, the term “composition for topical use” means a composition which may be applied to keratinous matters such as skin including the scalp, hair, eyelash, nails, and mucous membranes such as lips.

In the context of the present invention, the term “sequestering agent” means a compound capable of combining or forming a complex with transition metals.

Among the sequestering agents which may be used in the composition of the invention, mention may be made, for example, of ethylenediaminetetraacetic acid (EDTA) and its salts, such as the disodium salt of ethylenediaminetetraacetic acid (disodium EDTA); phosphonic derivatives such as hexamethylenediaminetetra(methylenephosphonic acid), ethylenediaminetetra(methylenephosphonic acid), 1-hydroxyethylidene-1,1-diphosphonic acid, aminotris(methylenephosphonic acid), diethylenetriaminepentakis(methylenephosponic acid) and salts thereof and in particular the sodium salts thereof, for instance the pentasodium salt of ethylenediaminetetrakis(methylenephosphonic acid); polymers and in particular polyamines such a polyalkylenepolyamines and derivatives thereof, and in particular polyethyleneimine, and dendrimers with chelating activity; proteins such as spermine, spermidine, transferrin and ferritin; carboxylic acids, for instance phytic acid, citric acid, malic acid, nitriloacetic acid, fumaric acid, tartaric acid, succinic or oxalic acid; desferrioxamine mesylate; and mixtures of these sequestering agents.

Preferably, the sequestering agent is chosen from hexamethylenediaminetetra(methylenephosphonic acid), ethylenediaminetetra(methylenephosphonic acid), 1-hydroxyethylidene-1,1-diphosphonic acid, aminotris(methylenephosphonic acid), diethylenetriaminepentakis(methylenephosponic acid), salts thereof and in particular the sodium salts thereof, and mixtures thereof.

For example, it is advantageously possible to use ethylenediaminetetrakis(methylenephosphonic acid), sold by the company Monsanto under the name Dequest 2041, and the pentasodium salt thereof which is sold under the name Dequest 2046 by the company Monsanto.

According to one preferred embodiment of the present invention, the composition according to the invention may be substantially free of antioxidants. In the context of the present invention, the term “substantially free of antioxidants” means that the composition contains less than 2% by weight, preferably less than 0.5% by weight, more preferably less than 0.1% by weight, even more preferably less than 0.01% by weight, better still 0% by weight, of antioxidant, based on the total weight of the composition. In a particularly preferred embodiment, the compositions of the present invention contain no added antioxidant. In the context of the present invention, the term antioxidant is to be understood as including those compounds which are conventionally used to impede the oxidation of cosmetic compositions and foods such BHT, tocopherol, and tocopheryl acetate

The glycoglycerides used in the composition of the invention are esters comprising one or two alkyl and/or alkenyl chains, and they may be obtained by extraction or by synthesis. Those obtained by extraction are especially plant extracts. The synthetic products may be obtained in particular by esterification of a sugar comprising at least one carboxylic acid function with a monoglyceride or a diglyceride. The sugars concerned are oses (monosaccharides), oligoholosides (oligosaccharides) and polyholosides (polysaccharides) that are highly depolymerized, preferably with a molecular mass of less than 2,000 g/mol. Non-limiting examples of these sugars which may be mentioned are glucose, sucrose, trehalose, sorbitol, lactose, fructose, xylose and maltose.

The alkyl and/or alkenyl chains of the glycoglyceride preferably comprise from 10 to 22 carbon atoms and are chosen, for example, from the alkyl and/or alkenyl chains in capric, lauric, myristic, palmitic, stearic, arachidic, behenic, palmitoleic, oleic, linoleic, linolenic, gadoleic, arachidonic, and erucic acids.

According to one preferred embodiment of the invention, the glycoglycerides used are galactolipids, in particular digalactosyl diglyceride or 1-O-[6-O-(α-D-galactopyranosyl)-β-D-galactopyranosyl]-2,3-di-O-acyl-D-glyceritol (DGDG) which may be in hydrogenated or non-hydrogenated form; monogalactosyl diglyceride (MGDG) and mixtures thereof. These compounds exist in the natural state in plants and more particularly in plant membranes. They may be obtained by extracting these plants, for example from cereals or spinach. They are described, for example, in published European patent applications EP-A-9842 and EP-A-249 561, which are incorporated herein in their entirety by reference. The DGDG and MGDG supplied by the companies Serdary Research Laboratories or Doosan may be used in particular. DGDG may also be prepared from fresh spinach leaves, according to the process of Example 1 of published European patent application EP-A-249 561, or from cereals, according to the process described in published European patent application EP-9842.

DGDG has the structure (I) below:

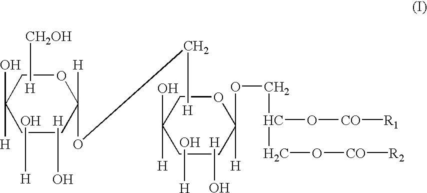

in which R₁ and R₂ may be identical or different and each independently represents an alkyl and/or alkenyl residue containing from 10 to 22 carbon atoms, such as the alkyl and alkenyl residues in capric, lauric, myristic, palmitic, stearic, arachidic, behenic, palmitoleic, oleic, linoleic, linolenic, gadoleic, arachidonic, or erucic acids.

These galactolipids may be used pure or as mixtures containing them in larger or smaller proportions. Mixtures containing at least 10% of DGDG relative to the polar lipids of the the mixture are preferably used.

The amount of sequestering agent(s) varies according to the amount of glycoglyceride(s) present. It may range, for example, from 0.01% to 20% by weight, preferably from 0.5% to 10% by weight, based on the total weight of the combination of sequestering agent(s) and glycoglyceride(s), the glycoglyceride(s) representing the difference to 100% by weight The weight ratio of the sequestering agent to the glycoglyceride (on an active material basis) preferably ranges from 1×10⁻⁴ to 0.2 and better still from 0.5×10⁻² to 0.1.

The composition according to the present invention comprising the combination of a glycoglyceride and a sequestering agent constitutes a composition for topical use and in particular a cosmetic and/or dermatological composition, and it comprises a physiologically acceptable medium. In the context of the present invention, the expression “physiologically acceptable medium” means a non-toxic medium which may be applied to the skin (including the inside of the eyelids), the hair, the nails or the lips of human beings.

In the composition according to the present invention intended for topical application, the amount of glycoglyceride(s) may range, for example, from 0.1% to 10% by weight (on an active material basis), preferably from 0.5% to 5% by weight, based on the total weight of the composition, and the amount of sequestering agent(s) may range, for example, from 0.001% to 5% by weight, preferably from 0.01% to 2% by weight, based on the total weight of the composition.

The composition of the present invention which comprises the combination of glycoglyceride and sequestering agent may be in any form normally used in cosmetics and dermatology, and may especially be in the form of a dispersion of the lotion type optionally containing two phases, an oil-in-water (O/W) or water-in-oil (W/O) emulsion, or a triple emulsion (W/O/W or O/W/O) or a vesicular dispersion of ionic type (liposomes) and/or nonionic type. These compositions may be prepared according to the usual methods.

The composition according to the present invention may comprise an oily phase (or fatty phase) based on one or more oils chosen from oils of plant origin (jojoba oil, avocado oil, sesame oil, sunflower oil, corn oil, soybean oil, safflower oil, or grapeseed oil), mineral oils (petroleum jelly, hydrogenated or nonhydrogenated isoparaffins, isohexadecane, or squalane), synthetic oils (parleam, isopropyl myristate, cetearyl octanoate, polyisobutylene, ethylhexyl palmitate, or alkyl benzoates), volatile or non-volatile silicone oils such as polydimethylsiloxanes (PDMSs) and cyclodimethylsiloxanes or cyclomethicones, and fluoro oils or fluorosilicone oils, and also mixtures of these oils. The oily phase may also contain other fatty constituents, such as fatty alcohols, for instance stearyl alcohol, cetyl alcohol and a mixture thereof (cetearyl alcohol); fatty acids; waxes; silicone gums.

The composition according to the present invention may comprise an aqueous phase which may contain, besides water, water-soluble or water-dispersible solvents and additives. Solvents which may be mentioned, for example, are linear or branched monoalcohols containing from 1 to 8 carbon atoms, for instance ethanol, propanol, isopropanol, butanol and isobutanol; polyethylene glycols containing from 4 to 80 ethylene oxides; polyols, for instance glycerol, propylene glycol, isoprene glycol and butylene glycol.

According to one particular embodiment of the present invention, the composition of the invention is in the form of a W/O emulsion. The aqueous phase of the W/O emulsion may vary within a wide range and may constitute up to 98% by weight of the total weight of the composition. It generally represents from 50% to 90% by weight relative to the total weight of the composition, and the oily phase generally represents from 0.5% to 40% by weight relative to the total weight of the composition.

The glycoglycerides are preferably introduced into the oily phase of the composition containing them, while the sequestering agent is generally introduced into the aqueous phase.

The compositions of the present invention, when in the form of emulsions, may contain surfactants commonly used for preparing emulsions. As surfactants which may be used for preparing W/O emulsions, mention may be made, for example, of alkyl esters or ethers of sorbitan, of glycerol or of sugars, and alkyldimethicone copolyols.

When the composition of the present invention contains surfactants other than glycoglycerides, these surfactants are present in an amount ranging from 0.1% to 50% by weight relative to the weight of glycoglycerides or of the mixture of lipids containing glycoglycerides.

The compositions according to the present invention find application in a large number of treatments and especially cosmetic treatments of the skin, including the scalp, the hair, the nails and/or mucous membranes, in particular for caring for, cleansing and/or making up and/or photoprotecting the skin, the hair and/or the lips or mucous membranes.

Thus, one embodiment of the present invention is a method for treating, protecting, caring for, removing make-up from and/or cleansing the skin, the lips and/or the hair, and/or for making up the skin and/or the lips, by applying an effective amount of the present composition.

Another embodiment of the present invention is a cosmetic method for treating the skin, including the scalp, the hair and/or the lips, by applying a cosmetic composition as defined above to the skin, the hair and/or the lips.

As noted above, the present compositions may be used for treating, protecting, caring for, removing make-up from, and/or cleansing the skin, the lips, the scalp, and/or the hair, and/or of making up the skin and/or the lips by applying the composition to the skin, lips, scalp, and/or hair. When using the present composition in such a manner, the composition is suitably applied to the skin, lips, scalp, and/or hair in an amount of 0.1 to 5 mg/cm², preferably 0.5 to 4 mg/cm², more preferably 1 to 3 mg/cm² (these numbers being approximate). The amount is generally 2 mg/cm².

In a known manner, the composition of the invention may also contain adjuvants that are common in cosmetics and/or dermatology, such as electrolytes, for instance magnesium sulfate, active agents, preserving agents, antioxidants, solvents, hydrophilic or lipophilic texturing agents and gelling agents, fragrances, fillers and in particular mineral or organic particles, bactericides, odor absorbers, colorants and lipid vesicles. The amounts of these various adjuvants are those used conventionally in the field under consideration, and, for example, from 0.01% to 20% of the total weight of the composition. Depending on their nature, these adjuvants may be introduced into the fatty phase or into the aqueous phase. In addition, these adjuvants and their concentrations must be such that they do not modify the property desired for the composition of the invention.

Active agents which may mentioned in particular, besides those indicated above, are moisturizers and, for example, protein hydrolysates and polyols such as glycerol, glycols, for instance polyethylene glycols, and sugar derivatives; natural extracts; procyannidol oligomers; vitamins; urea; caffeine; depigmenting agents such as kojic acid and caffeic acid; β-hydroxy acids such as salicylic acid and its derivatives; α-hydroxy acids such as lactic acid and glycolic acid; retinoids such as carotenoids; screening agents; and mixtures thereof.

The active agents(s) may be present, for example, in a concentration ranging from 0.01% to 20% by weight, preferably from 0.1% to 5% by weight, better still from 0.5% to 3% by weight, of the total weight of the composition.

Other features of the invention will become apparent in the course of the following descriptions of exemplary embodiments which are given for illustration of the invention and are not intended to be limiting thereof.

### EXAMPLES

In the examples below, the amounts therein are given as percentages by weight, except where otherwise mentioned.

1. Example according to the invention: W/O emulsion

| | |
|---|---|
| A. Oily phase | |
| Galactolipids (DGDG supplied by the company | 2.5% |
| Doosan) | |
| Isohexadecane | 7.7% |
| Squalane | 5.8% |
| Cyclomethicone | 3.9% |
| Preserving agents | 0.1% |
| B. Aqueous phase | |
| MgSO₄.7H₂0 | 0.8% |
| Preserving agents | 0.6% |
| Fragrance | 0.2% |
| Dequest 2046 | 0.1% |
| Water | qs 100% |

A fluid cream which is stable for at least two months at 45° C. is obtained.

2. Comparative example: W/O emulsion

| | |
|---|---|
| A. Oily phase | |
| Galactolipids (DGDG supplied by the company | 2.5% |
| Doosan) | |
| Isohexadecane | 7.7% |
| Squalane | 5.8% |
| Cyclomethicone | 3.9% |
| Preserving agents | 0.1% |
| BHT | 0.07% |
| Tocopheryl acetate | 0.75% |
| B. Aqueous phase | |
| MgSO₄.7H₂0 | 0.8% |
| Preserving agents | 0.6% |
| Fragrance | 0.2% |
| Water | qs 100% |

BHT and tocopheryl acetate are antioxidants.

3. Comparative test

The respective degrees of yellowing of the compositions of the example according to the invention and of the comparative example were measured using a chromameter after storage for two months at 45° C. The measurement was carried out directly on the flask containing the composition using a CR-300 chromameter from the company Minolta (technical parameters: measuring geometry: d/0°, size of the measuring area: 8 mm, source: xenon arc lamp, illuminant D65). The color measured is characterized by 3 coordinates (L, a, b) in the L*a*b space in which L represents the clarity, a represents the red-green axis (a negative value of a corresponds to green and a positive value of a corresponds to red) and b represents the yellow-blue axis (a negative value of b corresponds to blue, and a positive value of b corresponds to yellow). If a=b=0, the shade is gray.

Moreover, the rancid odor was evaluated by the chemist who prepared the composition, and also by a perfume expert. The results are shown in Table 1 below.

TABLE 1

| | |
|---|---|
| Example according | |
| to the invention | Comparative example |

| | Time | After two months at | Time | After two months at |
|---|---|---|---|---|
| | zero | 45° C. | zero | 45° C. |
| L | 77.72 | 76.16 | 81.02 | 81.49 |
| a | −0.51 | −0.64 | −1.28 | −2.26 |
| b | 3.29 | +3.32 | +4.55 | +8.67 |
| Odour | None | none | none | strong |

This table shows that, for the comparative example, the green and blue components increase very significantly at the end of the storage period, thus inducing an increase in the overall yellow color. Furthermore, a strong rancid odor developed, indicating degradation of the galactolipid. This is unacceptable for the marketing of such a composition. On the other hand, the composition according to the invention has no rancid odor, and its color is unchanged: its chromatic characteristics have not changed and its color has remained perfectly stable.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A composition for topical use, comprising in a physiologically acceptable medium, at least one glycoglyceride and at least one sequestering agent.

2. The composition of claim 1, wherein said sequestering agent is selected from the group consisting of ethylenediaminetetraacetic acid and its salts; phosphonic derivatives and salts thereof; polyamine polymers and derivatives thereof; proteins; carboxylic acids; desferrioxamine mesylate; and mixtures thereof.

3. The composition of claim 1, wherein said sequestering agent is selected from the group consisting of hexamethylenediaminetetra(methylenephosphonic acid), ethylenediaminetetra(methylenephosphonic acid), 1-hydroxyethylidene-1,1-diphosphonic acid, aminotris(methylenephosphonic acid), diethylenetriaminepentakis(methylenephosponic acid), salts thereof, and mixtures thereof.

4. The composition of claim 1, wherein said glycoglyceride is obtained by synthesis or by extraction.

5. The composition of claim 1, wherein said glycoglyceride is extracted from a plant.

6. The composition of claim 1, wherein said the glycoglyceride is obtained by esterification of a sugar comprising at least one carboxylic acid function with a monoglyceride or a diglyceride.

7. The composition of claim 1, wherein said glycoglyceride is a galactolipid.

8. The composition of claim 7, wherein said galactolipid is selected from the group consisting of hydrogenated or non-hydrogenated digalactosyl diglyceride and monogalactosyl diglyceride, and mixtures thereof.

9. The composition of claim 1, wherein said sequestering agent and said glycoglyceride are present in a weight ratio of said sequestering agent to said glycoglyceride of from 1×10⁻⁴ to 0.2.

10. The composition of claim 1, wherein said glycoglyceride is present in an amount of from 0.1% to 10% by weight relative to the total weight of the composition.

11. The composition of claim 1, wherein said sequestering agents is present in an amount of from 0.001% to 5% by weight relative to the total weight of the composition.

12. The composition of claim 1, which it is in the form of a dispersion, an oil-in-water or water-in-oil emulsion, a triple emulsion or a vesicular dispersion of ionic and/or nonionic type.

13. The composition of claim 1, which is in the form of a water-in-oil emulsion.

14. A method for treating, protecting, caring for, removing make-up from and/or cleansing the skin, the lips and/or the hair, and/or for making up the skin and/or the lips, comprising applying to the skin, the lips and/or the hair of a subject in need thereof an effective amount of a composition, comprising in a physiologically acceptable medium, at least one glycoglyceride and at least one sequestering agent.

15. The method of claim 14, wherein said sequestering agent is selected from the group consisting of hexamethylenediaminetetra(methylenephosphonic acid), ethylenediaminetetra(methylenephosphonic acid), 1-hydroxyethylidene-1,1-diphosphonic acid, aminotris(methylenephosphonic acid), diethylenetriaminepentakis(methylenephosponic acid), salts thereof, and mixtures thereof.

16. A method for treating the skin, including the scalp, the hair and/or the lips, comprising applying to the skin, the lips and/or the hair of a subject in need thereof an effective amount of a composition, comprising in a physiologically acceptable medium, at least one glycoglyceride and at least one sequestering agent.

17. The method of claim 16, wherein said sequestering agent is selected from the group consisting of hexamethylenediaminetetra(methylenephosphonic acid), ethylenediaminetetra(methylenephosphonic acid), 1-hydroxyethylidene-1,1-diphosphonic acid, aminotris(methylenephosphonic acid), diethylenetriaminepentakis(methylenephosponic acid), salts thereof, and mixtures thereof.

18. A method of stabilizing a composition for topical use, said composition comprising at least one glycoglyceride, said method comprising adding at least one sequestering agent to said composition.

19. The method of claim 18, wherein said sequestering agent is selected from the group consisting of hexamethylenediaminetetra(methylenephosphonic acid), ethylenediaminetetra(methylenephosphonic acid), 1-hydroxyethylidene-1,1-diphosphonic acid, aminotris(methylenephosphonic acid), diethylenetriaminepentakis(methylenephosponic acid), salts thereof, and mixtures thereof.

20. The method of claim 18, wherein said sequestering agent is added to said composition in an amount of from 0.01% to 20% by weight, based on the total weight of sequestering agent and glycoglyceride.
